(19) United States
(12) Patent Application Publication
(10) Pub. No.: US 2006/0183889 A1
(43) Pub. Date: Aug. 17, 2006

(54) Alkyl phosphine detergent products and extraction processes

(76) Inventor: Roumen A. Bogoev, San Marcos, CA (US)

Correspondence Address: BIOTECHNOLOGY LAW GROUP;C/O PORTFOLIOIP, PO BOX 52050, MINNEAPOLIS, MN, 55402, US

(21) Appl. No.: 11/324,816

(22) Filed: Jan. 4, 2006

Related U.S. Application Data
(60) Provisional application No. 60/641,202, filed on Jan. 4, 2005.

(51) Int. Cl. C07K 1/10

(52) U.S. Cl. 530/412

(57) ABSTRACT

Provided herein are compositions and kits comprising alkylphosphine oxide detergents, and methods for the use thereof for the extraction of component molecules from cells. These compositions, kits and methods can provide efficient extraction of cellular components in a mild environment. While applicable to cell-associated molecules generally, the methods and compositions described herein are sometimes used to extract and purify polypeptides from cells or cell extracts, while maintaining a mild pH.

### RELATED APPLICATION

This patent application claims the benefit of U.S. Provisional Patent Application No. 60/641,202 filed on 04 Jan. 2005, entitled “Alkyl Phosphine Detergent Products And Extraction Processes,” naming Roumen Anguelov Bogoev as an inventor, and designated by attorney docket no. INV-1004-PV. The content and subject matter of this patent application is hereby incorporated by reference in its entirety, including all text and drawings.

### FIELD OF THE INVENTION

The present invention relates in various parts to compositions and methods to facilitate the extraction of molecules from cells.

### BACKGROUND

The following discussion of the background of the invention is merely provided to aid the reader in understanding the invention and is not admitted to describe or constitute prior art.

A variety of procedures have been employed to facilitate the preparation of molecules such as proteins, polypeptides, nucleic acids, and various complexes thereof, from association with organisms (such cell-associated proteins may be located, for example, within cells, associated with cell membranes, or located within or associated with extracellular spaces or structures such as cell walls). As an initial step, the contents of the organism or cells of interest are typically liberated, e.g., by lysis, rupture and/or permeabilization of the cells. Following this release of contents, one or more desired species may be purified from the cell extract, often by a series of chromatographic, precipitation, and/or selective binding steps.

Several approaches have proven useful in accomplishing the release of such cell-associated molecules from cells. Included among these are chemical lysis or permeabilization, physical methods of disruption, or a combination of chemical and physical approaches. Chemical methods of disruption of the bacterial cell wall generally involve treatment of cells with organic solvents, chaeotropes, antibiotics, detergents, and/or enzymes. Physical methods generally include osmotic shock, drying, shear forces (employing, for example, bead mills or blenders), temperature shock, ultrasonic disruption, or some combination of the above (e.g., a French press generates both shear forces and an explosive pressure drop). Other approaches combine chemical and physical methods of disruption generally involve enzymatic (e.g., lysozyme) treatment followed by sonication or pressure treatment to maximize cell disruption and protein release.

As noted above, detergents are widely used in the art to solubilize membranes, to enhance permeabilization effects of various chemical agents, and for disruption of the bacterial cell walls. See, e.g., U.S. Pat. Nos. 5,625,053, 6,174,707, and 6,821,752. In particular, detergent use can facilitate the preparation of cell-associated proteins, and various approaches have been used in the initial steps of processes for the purification of a variety of cytosolic and membrane proteins, including natural and recombinant proteins from mesophilic organisms such as Escherichia coli, from thermophilic bacteria and archaea, and from eukaryotic cells. However, even when detergents are not employed during the initial steps of fractionation, they are often added subsequently in order to facilitate fractionation of the cell extract into various subportions. In addition, the addition of detergents to an extraction procedure can help to stabilize certain extracted proteins. For example, Simpson et al., Biochem. Cell Biol. 68: 1292-6 (1990) discloses purification of a DNA polymerase that is stabilized by additives such as Triton X-100.

Each reference cited in the preceding section is hereby incorporated by reference in its entirety, including all tables, figures, and claims.

### SUMMARY

Provided are compositions and methods useful for facilitating the extraction of molecules from cells. These compositions and methods comprise the use of one or more alkylphosphine oxide (“APO”) detergents having the following general formula: 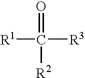
where R¹ and R² are each independently selected from the group consisting of straight or branched chain C₁₋₆ alkyl, straight or branched chain C₁₋₆ alkenyl, and straight or branched chain C₁₋₆ alkynyl, each optionally substituted with from 1 to 4 substituents independently selected from the group consisting of halogen, trihalomethyl, C₁₋₆ alkoxy, —NO₂, —NH₂, —OH, —COOR′, where R′ is H or lower alkyl, —CH₂OH, or —CONH₂; and R³ is C₈-C₁₆ straight or branched chain alkyl, alkenyl, or alkynyl. Such detergents are referred to generally herein as “APO detergents.”

As used herein, the term alkyl refers to optionally substituted straight or branched chain hydrocarbon groups joined by single carbon-carbon bonds; the term alkenyl refers to optionally substituted straight or branched chain hydrocarbon groups containing at least one carbon-carbon double bond; and the term alkynyl refers to optionally substituted straight or branched chain hydrocarbon groups having at least one carbon-carbon triple bond. The term lower alkyl refers to optionally substituted straight or branched chain hydrocarbon groups joined by single carbon-carbon bonds and comprising from 1-6 carbon atoms.

One or more APO detergents may be used alone or in combination with one or more additional detergents, enzymes, or physical extraction procedures to facilitate recovery of molecules, particularly proteins or other polypeptides, from cells; to help stabilize such molecules recovered from cells; and/or to facilitate recovery and/or stability of such molecules from cell extracts.

In a first aspect, provided are compositions comprising one or more APO detergents in a buffered aqueous solution having a pH of between about 3 and about 10. APO detergent concentrations sometimes are between about 0.2% to about 4% weight/volume, and sometimes between about 0.5% to about 2% weight/volume.

APO detergents for use in forming such buffered aqueous solutions often include those in which R³ is selected from the group consisting of C₈, C₉, C₁₀, C₁₁, and C₁₂, alkyl, alkenyl, or alkynyl, sometimes as a straight (linear) alkyl chain. R¹ and R² groups often are independently selected from the group consisting of methyl, ethyl, 1-propyl, 2-propyl, 1-butyl, 2-butyl, 2-methyl-1-propyl, tert-butyl, 1-pentyl, 2-pentyl, 3-pentyl, hydroxymethyl, 2-hydroxyethyl, 2-hydroxypropyl, 3-hydroxypropyl and 2,3-dihydroxypropyl. Dimethyldecylphosphine oxide often is utilized as an APO detergent.

In principle, any buffering agent(s) can be used in the aqueous solution to arrive at a final composition, depending on the desired pH of the final medium. Various buffered aqueous solutions sometimes have a pH of between about 6 and about 9, often between about 6.5 and about 7.5, and sometimes between about 7 and about 7.5. Buffering agents sometimes are selected from the group consisting of ACES, ADA, BES, bicine, bis-tris, CAPS, CHES, diethylmalonate, glycylglycine, glycinamide HCl, HEPES, HEPPS, imidazole, MES, MOPS, MOPSO, PIPPS, PIPES, POPSO, TAPSO, TES, tricine, tris, phosphate, bicarbonate, and borate. This list is exemplary and not limiting. Buffering agent concentrations often are less than 2 M, sometimes are about 0.2 M, 0.1 M, 0.05 M, 0.05 M, 0.02 M, 0.01 M, 0.005, 0.001, and 0.0001 M, and buffered aqueous solutions often have a pH selected from the group consisting of 6.5, 6.75, 7, 7.25, 7.5, 7.75, 8, 8.25, 8.5, 8.75, and 9.

In addition to one or more APO detergents and one or more buffering agents, the compositions of the present invention may further comprise one or more additional detergents, one or more kosmotropes, one or more chaotropes, and/or one or more enzymes.

Thus, in various embodiments, one or more additional detergents may be included in the buffered aqueous solutions comprising an APO detergent. Suitable non-APO detergents used for this purpose include, but are not limited to, anionic detergents such as sodium n-dodecyl sulfate (SDS); dihydroxy or trihydroxy bile acids (and their salts), such as cholic acid (sodium cholate), deoxycholic acid (sodium deoxycholate), taurodeoxycholic acid (sodium taurodeoxycholate), taurocholic acid (sodium taurocholate), glycodeoxycholic acid (sodium glycodeoxycholate), glycocholic acid (sodium glycocholate); cationic detergents such as cetyl trimethyl-ammonium bromide (CTAB); non-ionic detergents such as the polyoxyethylenes NP-40, TRITON® X-100, TRITON® X-114, C₁₂E₈, C₁₂E₉, GENAPOL® X-080, GENAPOL® X-100, LUBROL® PX, BRIJ® 35, TWEEN® 20, and TWEEN® 20; alkyl glycosides such as dodecyl-β-D-maltoside (“dodecyl maltoside”), n-nonyl-β-D-glucopyranoside, n-octyl-β-D-glucopyranoside (“octyl glucoside”), n-heptyl-β-D-glucopyranoside, and n-hexyl-β-D-glucopyranoside; alkylamine oxides such as lauryl dimethylamine oxide (LDAO); and zwitterionic detergents, such as CHAPS, CHAPSO, n-dodecyl-N,N-dimethylglycine, and ZWITTERGENTS® 3-08, 3-10, 3-12, 3-14, and 3-16. This list is exemplary and not limiting.

In certain embodiments, the compositions of the present invention may further comprise one or more kosmotropes and/or chaotropes. Large singly charged ions with low charge density (e.g. SCN⁻, H₂PO₄⁻, HSO₄⁻, HCO₃⁻, I⁻, Cl⁻, NO₃⁻, NH₄⁺, Cs⁺, K⁺, (NH₂)₃C⁺ (guanidinium) and (CH₃)₄N⁺ (tetramethylammonium) ions) are considered ionic chaotropes, whereas small or multiply-charged ions, with high charge density, (e.g. SO₄²⁻, HPO₄²⁻, Mg²⁺, Ca²⁺, Li⁺, Na⁺, H⁺, OH⁻ and HPO₄²⁻) are considered ionic kosmotropes. Thus, the present invention contemplates the use of various salts of these ionic species in preparing the compositions of the present invention (e.g., those having sodium, potassium, magnesium, calcium, and/or ammonium as counterions to the foregoing negatively charged ions, and chloride, sulfate, and/or bromide as counterions to the foregoing positively charged ions). Nonionic kosmotropes include polyhydric alcohols, trehalose, glucose, trimethylamine N-oxide, glycine betaine, ectoine, proline and various other zwitterions, whereas nonionic chaotropes include guanidinium chloride, and urea (at concentrations often between 1M and 8 M). The foregoing lists are exemplary and not limiting.

Likewise, in other embodiments, one or more enzymes may be included in the buffered aqueous APO detergent compositions of the present invention. Enzymes sometimes are those that degrade a cellular component, sometimes including those that hydrolyze cell wall structures, proteins, nucleic acids, and/or oligosaccharides, etc. For example, lysozyme, β-1,3-glucanase, zymolase, lysostaphin, and cellulase digestion are frequently used with bacteria, yeast, and plant cells to dissolve a coat, capsule, capsid, cell wall, or other structure not easily sheared by mechanical methods alone. Similarly, RNases and/or DNases are often included by the artisan in protein extraction reagents. The foregoing lists are exemplary and are not limiting.

In still other embodiments, one or more additional reagents that are compatible with aqueous solutions may be included in the buffered aqueous APO detergent compositions of the present invention. Additional reagents sometimes include reducing agents (e.g., DTT and/or 2-mercaptoethanol), agents providing osmotic balance (e.g., sugar alcohols such as glycerol and sorbitol, polyalkylene oxides such as more poly(ethylene oxide), and/or dextrans), and/or organic solvents (e.g., methanol, dimethylformamide, and/or dimethyl sulfoxide). The foregoing lists are exemplary and are not limiting.

As discussed above, the compositions of the present invention find particular utility as reagents for preparation of cell-associated molecules such as proteins, polypeptides, nucleic acids, and various complexes thereof, from organisms. Thus, in a related aspect, the invention relates to methods for extracting one or more molecules of interest from cells comprising contacting cells, or an extract obtained therefrom, with one or more APO detergents.

In various embodiments, the methods described herein are used to extract one or more molecules of interest from microorganisms. Exemplary genera are selected from the group consisting of Escherichia, Bacillus, Salmonella, Pseudomonas, Streptomyces, Staphylococcus, Clostridia, Cryptosporidia, Entamoeba, Thermus, Pyrococcus, Thermococcus, Aquifex, Sulfolobus, Thermoplasma, Thermoanaerobacter, Rhodothermus, Methanococcus, and Thermotoga. Microorganisms are E. coli in some embodiments. The foregoing lists are exemplary and are not limiting.

In alternative embodiments, the methods described herein are used to extract one or more molecules of interest from yeast cells. Exemplary genera are selected from the group consisting of Saccharomyces, Pichia, and Kluveromyces. Yeast sometimes are S. cerevisiae and P. pastoris. The foregoing lists are exemplary and are not limiting.

In still other embodiments, the methods described herein are used to extract one or more molecules of interest from eukaryotic cells. Suitable eukaryotic cells include, but are not limited to, non-human tissue culture cells and human tissue culture cells. Eukaryotic cells include insect cells, HeLa cells, Chinese hamster ovary cells (CHO cells), African green monkey kidney cells (COS cells), human 293 cells, and murine 3T3 fibroblasts. Other suitable cells include monocotyledonous and dicotyledonous plant cells, such as cells from soybean, cotton, rice, oilseed rape, flax, kiwi fruit, tomato, sugar beet, grape, apple, tobacco, sunflower, and potato. The foregoing lists are exemplary and are not limiting.

In certain embodiments, the methods described herein are used to extract one or more nucleic acids (RNA and/or DNA), polypeptides, proteins, and/or enzymes from the prokaryotic and/or eukaryotic cells contacted with the buffered aqueous APO detergent compositions of the present invention. In some embodiments, the methods of the present invention are used to extract one or more exogenous polypeptides expressed in a host cell using an expression vector encoding the polypeptide(s) of interest. Methods for expressing polypeptides in host cells are well known in the art. See, e.g., Hodgson, Expression Systems: A User's Guide Bio/Technology 11, 887-893, 1993. Typical host cells include insect cells (See, e.g., Luckow et al., Bio/Technology (1988) 6: 47-55, 1988; Baculovirus Expression Vectors: A Laboratory Manual, O'Rielly et al. (Eds.), W. H. Freeman and Company, New York, 1992; and U.S. Pat. No. 4,879,236; bacteria (see, e.g, Sambrook, et al., Molecular Cloning: A Laboratory Manual, Cold Spring Harbor Laboratory, Cold Spring Harbor, N.Y., 1989; Gene Expression in Recombinant Microorganisms, A. Smith, ed., Marcel Dekker, Inc. New York, 1994); yeast (See, e.g., Pichia Protocols, in Methods in Molecular Biology, vol. 103, Higgins and Cregg, eds., Humana Press, NJ, 1998); and plant cells (see, e.g, Kusnadi et al., Production of Recombinant Proteins in Transgenic Plants: Practical Considerations, Biotech. and Bioeng. 5: 473-84, 1997). The foregoing lists are exemplary and are not limiting.

In some embodiments, the exogenous polypeptide(s) to be extracted from cells comprise(s) an amino acid sequence tag, sometimes to provide affinity enrichment of the polypeptide of interest by binding of the tag to a cognate binding partner. Examples of such affinity tags commonly used in the art include, but are not limited to, sequences providing a polyhistidine (or other metal-binding sequence), FLAG, V5, c-myc, influenza hemaglutinin, bacterial glutathione-S-transferase, maltose binding protein, thioredoxin, β-galactosidase, VSV-glycoprotein, and/or cysteine-rich sequence (e.g., the Lumio™ tag). Similarly, exogenous polypeptide(s) to be extracted from cells may comprise an amino acid sequence tag to provide detection of the polypeptide of interest in a manner other than affinity binding. For example, various fluorescent polypeptides (e.g., green fluorescent protein and its many color variants) may be expressed as fusions with a polypeptide of interest. More than one tag may be present on an exogenous polypeptide, such regions may sometimes be provided at the amino terminal and/or carboxyl terminal end of the polypeptide of interest, and such regions may optionally contain a protease-sensitive site to provide for removal of the tag from the polypeptide of interest as desired by the artisan.

The step of contacting cells with an APO detergent may be combined with one or more other methods for extracting cellular components. For example, cells may also be subjected to one or more physical methods (which, as discussed above include osmotic shock, drying, shear forces, temperature shock, ultrasonic disruption, or some combination of these) for cell disruption. In various embodiments, the temporal order of these steps may be varied. Thus, cells may be subjected to physical disruption before, after, or at the same time as the cells are contacted with an APO detergent. Moreover, one or more separation methods (such as a centrifugation step, an affinity tag-based enrichment, etc.) may be employed to remove certain cellular components from the polypeptide(s) of interest. Separation method(s) may be combined with various other steps (e.g., physical disruption) described herein, and, again, the timing of such separation steps may be varied according to the protocol employed. Thus, cells may be initially disrupted by physical methods, subjected to one or more separation methods, and then contacted with an APO detergent. Alternatively, cells may be extracted using an APO detergent, and the resulting extract subjected to one or more separation methods without employing physical disruption.

Similarly, the various components described above for inclusion in the buffered aqueous APO detergent compositions may be contacted with the cells (or cell extract) in any order, according to the needs of the artisan. Accordingly, while a buffered aqueous APO detergent composition often is pre-formed and used to contact cells, cells may be suspended in a solution comprising one or more buffering agents described above, and then an APO detergent may be added; alternatively, cells may be contacted with an APO detergent, followed later by addition of one or more of buffering agents. Also, a solid form of the APO detergent may be provided and it may be formulated in a solution added to cells or a solution containing cells. Likewise, cells may be contacted with one or more of non-APO detergents, kosmotropes, chaotropes, enzymes, and/or additional reagents (sometimes selected from the non-limiting lists of these various components described above) before, after, or at the same time as the cells are contacted with an APO detergent. The relative order of the steps in an extraction method is appropriately left to the discretion of the artisan.

In another related aspect of the present invention, the buffered aqueous APO detergent compositions, and/or their component elements, described above, may be provided in kit form, in order to facilitate their use in the methods described herein. Such kits comprise one or more APO detergents, sometimes as a buffered aqueous solution as described herein.

In various embodiments, such kits further comprise one or more non-APO detergents, kosmotropes, chaotropes, enzymes, and/or additional reagents (sometimes selected from the non-limiting lists of these various components described above), either in separate containers from the APO detergent(s) of the invention or, often, as a component of a buffered aqueous APO detergent composition.

Such kits may further comprise one or more cells, sometimes transfection competent bacteria, for use in the methods described herein for expression of exogenous proteins; and/or one or more nucleic acids adapted for use in a polypeptide expression system. In the case that such nucleic acids are provided, such a nucleic acid sometimes encodes an amino acid sequence tag as described herein adapted for expression as part of a fusion polypeptide. Kits according to the invention may also comprise a reagent selected to provide binding to an affinity tag. Such reagents are sometimes coupled to a solid support.

### BRIEF DESCRIPTION OF THE DRAWINGS

FIG. 1 shows lysis of uninduced BL21 Star cells with various detergents as analyzed by polyacrylamide gel electrophoresis (PAGE).

FIG. 2 shows lysis of uninduced BL21 Star cells with APO detergents as analyzed by PAGE.

FIG. 3 shows lysis of BL21 AI cell cultures expressing green fluorescent protein (GFP).

FIG. 4 shows lysis of BL21 AI cells expressing beta-galactosidase.

### DETAILED DESCRIPTION

The present invention relates to various compositions and methods comprising certain alkylphosphine oxide detergents, finding use in the extraction of component molecules from cells. These compositions and methods can provide efficient extraction of cellular components in a mild environment.

While applicable to molecules present in cells generally, the methods and compositions described herein are sometimes used to extract and purify polypeptides from cells or cell extracts. Exemplary polypeptides include antibodies, enzymes, serum proteins (e.g., albumin), hormones (e.g., growth hormone, erythropoetin, insulin, etc.), cytokines, etc., and include both naturally occurring and exogenously expressed polypeptides. The term “polypeptide” as used herein refers to a molecule having a sequence of amino acids linked by peptide bonds. This term includes proteins, fusion proteins, oligopeptides, cyclic peptides, and polypeptide derivatives. Antibodies and antibody derivatives are, for purposes of the invention, treated as a subclass of the polypeptides and derivatives. The term protein refers to a polypeptide that is isolated from a natural source, or produced from an isolated cDNA using recombinant DNA technology, and that has a sequence of amino acids having a length of at least about 200 amino acids. A protein or polypeptide sometimes is intracellular (e.g., located in the nucleus, cytosol, or interstitial space of host cells) and sometimes is located in a cell membrane. The term “nucleic acids” as used herein shall be generic to polydeoxyribonucleotides (containing 2′-deoxy-D-ribose or modified forms thereof), to polyribonucleotides (containing D-ribose or modified forms thereof), and to any other type of polynucleotide which is an N-glycoside of a purine or pyrimidine bases, or modified purine or pyrimidine bases.

Provided herein is an aqueous solution which comprises a detergent and a buffer component at about pH 3 to about pH 10, wherein the detergent is of the following structure: 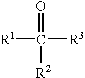
where R1 and R2 are independently selected from the group consisting of methyl, ethyl, 1-propyl, 2-propyl, 1-butyl, 2-butyl, 2-methyl-1-propyl, tert-butyl, 1-pentyl, 2-pentyl, 3-pentyl, hydroxymethyl, 2-hydroxyethyl, 2-hydroxypropyl, 3-hydroxypropyl and 2,3-dihydroxypropyl; and R3 is a C8 to C16 linear or branched alkyl moiety. In certain embodiments (a) R1 and R2 are methyl; (b) R3 is a C8 to C12 linear alkyl moiety; (c) R3 is a C9 or C10 linear alkyl moiety; (d) R3 is a C10 linear alkyl moiety; and (e) combinations of the foregoing. The pH sometimes is about 6 to about 9, sometimes is about 6 to about 8, can be about 6.5 to about 7.5, and may be about 7 or about 7.5. The detergent in certain embodiments is about 0.2% to about 4% by weight in the solution, and it can be about 0.5% to about 2% by weight in the solution. The buffer component is selected from the group consisting of a Tris buffer, a BisTris buffer, a phosphate buffer, a MOPS buffer, a MOPSO buffer, a PIPPS buffer and a HEPES buffer in some embodiments. The aqueous solution may comprise a denaturant, such as urea or sodium dodecyl sulfate, for example. In some embodiments, the solution comprises an agent that degrades a cell component. The foregoing agent may degrade cell walls (e.g., lysozyme), may degrade a nucleic acid (e.g., DNAse, RNAse) and/or may degrade an oligosaccharide (e.g., zymolyase). The aqueous solution may comprise one or more salts in certain embodiments (e.g., NaCl). In some embodiments, the solution comprises one or more other detergents, such as a Tween detergent or Triton X-100, for example. The solution sometimes comprises a viscosogen or osmolyte agent (e.g., glycerol or sucrose) and/or may contain a reducing agent, such as a thiol reducing agent (e.g., dithiothreitol, beta mercaptoethanol). In some embodiments, the solution comprises a protein detection agent. The detection agent sometimes comprises an arsenic atom, such as biarsenical fluorophore derivatives as adducts with EDT (1,2-ethanedithiol) (e.g., Lumio™ detection reagent; 4′,5′-bis(1,3,2-dithioarsolan-2-yl)fluorescein-(1,2-ethanedithiol)₂ or red-fluorescing biarsenical resorfin derivatives, for example, ReASH™ or Lumio™ Red, also commercially available). Other detection reagents described in U.S. Pat. Nos. 5,932,474, 6,054,271; 6,451,569; 6,008378; U.S. patent application publication no. 2003/0083373, and international patent application publication no. WO 99/21013, the disclosures of which are incorporated herein by reference in their entireties. The detection reagent can comprise a metal (e.g., a divalent metal such as copper, zinc cobalt, or nickel; e.g., InVision™ stain), or may comprise labeled affinity reagents such as antibodies or streptavidin. The solution in certain embodiments may comprise a protein capture agent. The protein capture agent may comprise an arsenic atom (e.g., 4′,5′-bis(1,3,2-dithioarsolan-2-yl)fluorescein-(1,2-ethanedithiol)₂) or metal (e.g., a divalent metal such as copper, zinc, nickel, or cobalt), or may comprise affinity reagents such as antibodies or other affinity reagents such as amylose, chitin, glutathione, biotin, avidin, streptavidin, etc. for binding protein sequences or domains, including sequences or domains of fusion proteins, tagged proteins, and labeled proteins. The protein capture agent often is in association with a solid support.

Also provided herein is a kit for protein extraction which comprises a container that includes a detergent of the following structure: 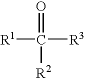
where R1, R2 and R3 are defined above. The kit may contain any component described above for aqueous solutions. A kit may contain a buffer reagent that buffers the pH of a solution between about pH 3 and about pH 10. The kit also may contain a container comprising a denaturant, such as urea or sodium dodecyl sulfate. In some embodiments, the kit includes a container comprising an agent that degrades a cell component, such as cell walls (e.g., lysozyme), a nucleic acid (e.g., a DNAse, RNAse) and/or an oligosaccharide (e.g., zymolyase). The kit in certain embodiments includes a container comprising another detergent (e.g., a Tween detergent, Triton X-100 detergent). The kit may includes a container comprising a viscosogen or osmolyte (e.g., glycerol or sucrose). In certain embodiments, the kit may include a container comprising a reducing agent. The reducing agent sometimes is a thiol reducing agent such as dithiothreitol or beta mercaptoethanol, for example. A kit in some embodiments includes a container comprising transfection competent bacteria (e.g., E. coli). Sometimes a kit includes a container that comprises a nucleic acid. The nucleic acid sometimes comprises a promoter operatively linked to an insertion region into which a target nucleotide sequence is capable of being positioned. The nucleic acid sometimes comprises a nucleotide sequence that encodes an amino acid tag adjacent to the insertion region. In certain embodiments, the amino acid tag is selected from the group consisting of glutathione S-transferase, maltose binding protein, V5 protein, a fluorescent protein, a polyhistidine sequence, and a cysteine-rich sequence. The amino acid sequence sometimes is CC-Xn-CC, where X is any amino acid and n is 1 to 3 (e.g., CCPGCC). In some embodiments, the kit includes a container comprising an agent that specifically binds to an amino acid tag. Such an agent sometimes is in association with a solid support. The agent sometimes is a metal (e.g., a bivalent metal such as nickel, cobalt or zinc), and in certain embodiments, the agent comprises two or more arsenic atoms. The agent fluoresces when it binds to an amino acid tag in some embodiments. A kit sometimes comprises a protein detection agent. The protein detection agent may comprise an arsenic atom (e.g., two or more arsenic atoms). Examples of such detection agents are Lumiotm detection reagent (e.g., 4′,5′-bis(1,3,2-dithioarsolan-2-yl)fluorescein-(1,2-ethanedithiol)₂). A detection agent in some embodiments may comprise a metal (e.g., a divalent metal such as copper, zinc, nickel, or cobalt). In certain embodiments, a kit comprises a protein capture agent. The protein capture agent sometimes comprises an arsenic atom or metal ion (e.g., 4′,5′-bis(1,3,2-dithioarsolan-2-yl)fluorescein-(1,2-ethanedithiol)₂, copper, zinc, cobalt, nickel, e.g., ProBond™ or NiNTA resins). The capture agent or detection agent sometimes is in association with a solid support. The association may be covalent or non-covalent (e.g., streptavidin-biotin) and may comprise affinity reagents such as antibodies or other affinity reagents such as amylose, chitin, glutathione, biotin, avidin, streptavidin, etc. for binding protein sequences or domains, including sequences or domains of fusion proteins, tagged proteins, and labeled proteins. Kits provided herein sometimes include instructions for contacting the detergent, and any other component, with cells.

Also provided is a composition which comprises cells and a detergent of the following structure: 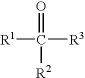
where R1, R2 and R3 are defined above, at about pH 3 to about pH 10. Provided also herein is a composition which comprises cells and an aqueous solution of any one of the preceding aspects at about pH 3 to about pH 10. The cells sometimes are bacteria (e.g., E. coli). The container sometimes further includes one or more contents of a kit described above. The composition sometimes is in a container.

Provided also herein is a method for extracting a peptide or protein from cells, which comprises: contacting the cells with a detergent in a solution at about pH 3 to about pH 10, where the detergent is of the following structure: 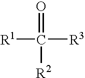
where R1 and R2 are independently selected from the group consisting of methyl, ethyl, 1-propyl, 2-propyl, 1-butyl, 2-butyl, 2-methyl-1-propyl, tert-butyl, 1-pentyl, 2-pentyl, 3-pentyl, hydroxymethyl, 2-hydroxyethyl, 2-hydroxypropyl, 3-hydroxypropyl and 2,3-dihydroxypropyl; and R3 is a C8 to C16 linear or branched alkyl moiety; whereby protein is extracted from the cells. The term “extracting” as used herein refers to releasing, separating or dissociating a protein or peptide from a cell, often by cell lysis or by perforating a cell membrane (i.e., introducing holes in a cell). Also provided is a method for isolating a target peptide or target protein from cells, which comprises: contacting the cells with a detergent of the following structure: 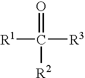
where R1 and R2 are independently selected from the group consisting of methyl, ethyl, 1-propyl, 2-propyl, 1-butyl, 2-butyl, 2-methyl-1-propyl, tert-butyl, 1-pentyl, 2-pentyl, 3-pentyl, hydroxymethyl, 2-hydroxyethyl, 2-hydroxypropyl, 3-hydroxypropyl and 2,3-dihydroxypropyl; and R3 is a C8 to C16 linear or branched alkyl moiety; and isolating the target peptide or target protein. In the latter method, the cells are in a solution having a pH of from about pH 3 to about pH 10. In certain embodiments, (a) R1 and R2 are methyl; (b) R3 is a C8 to C12 linear alkyl moiety; (c) R3 is a C9 or C10 linear alkyl moiety; (d) R3 is a C10 linear alkyl moiety and (e) combinations of the foregoing. The pH sometimes is about 6 to about 9, can be about 6 to about 8, sometimes is about 6.5 to about 7.5, and is about 7 or about 7.5 in certain embodiments. In some embodiments, the detergent is about 0.2% to about 4% by weight in the solution, and sometimes is about 0.5% to about 2% by weight in the solution. The solution sometimes comprises a buffer component, such as a Tris buffer, a BisTris buffer, a phosphate buffer, a MOPS buffer, a MOPSO buffer, a PIPPS buffer and a HEPES buffer, for example. In certain embodiments, the method comprises contacting the peptide or protein with a denaturant (e.g., urea, sodium dodecyl sulfate). The method sometimes comprises contacting the cells with an agent that degrades a cell component. The agent sometimes degrades cell walls (e.g., lysozyme), sometimes degrades a nucleic acid (e.g., a DNAse , a RNAse) and/or sometimes degrades an oligosaccharide (e.g., zymolyase). In some embodiments, cells are contacted with lysozyme when a target protein is about 70 kDa or larger. For smaller target proteins the inclusion of lysozyme is optional as it often does not affect the amount of protein extracted. Lysozyme is optional and often not required in extraction methods when lysozyme-expressing strains of E. coli, such as pLysS, are utilized. In certain embodiments, the method comprises contacting the protein or peptide with another detergent (e.g., a Tween detergent, Triton X-100 detergent). The method sometimes comprises contacting the protein or peptide with a viscosity enhancing agent (e.g., glycerol or sucrose). In some embodiments, the method comprises contacting the peptide or protein with a reducing agent, such as a thiol reducing agent (e.g., dithiothreitol, beta mercaptoethanol).

In certain method embodiments, the target protein or target peptide comprises an amino acid tag. The amino acid tag sometimes is selected from the group consisting of glutathione S-transferase, maltose binding protein, V5 protein, a fluorescent protein, a polyhistidine sequence, and a cysteine-rich sequence. A cysteine-rich sequence sometimes comprises the amino acid sequence CC-Xn-CC, where X is any amino acid and n is 1 to 3 (e.g., CCPGCC). Methods provided herein sometimes comprise contacting a target peptide or target protein with an agent that specifically binds to an amino acid tag. Such an agent sometimes is in association with a solid support. The agent sometimes comprises a metal (e.g., a bivalent metal such as nickel, cobalt or zinc), and in certain embodiments, the agent comprises two or more arsenic atoms. The agent fluoresces when it binds to an amino acid tag in some embodiments. In certain embodiments, the method comprises contacting the protein or peptide with a protein detection agent. The protein detection agent may comprise an arsenic atom (e.g., two or more arsenic atoms). Examples of such detection agents are Lumio™ detection reagent (e.g., 4′,5′-bis(1,3,2-dithioarsolan-2-yl)fluorescein-(1,2-ethanedithiol)₂). A detection agent in some embodiments may comprise a metal (e.g., a divalent metal such as copper, nickel, zinc or cobalt). In certain embodiments, the method comprises contacting a protein or peptide with a protein capture agent. The protein capture agent sometimes comprises an arsenic atom or metal ion (e.g., 4′,5′-bis(1,3,2-dithioarsolan-2-yl)fluorescein-(1,2-ethanedithiol)₂, copper, nickel, zinc, cobalt). The capture agent or detection agent sometimes is in association with a solid support. In certain method embodiments, the cells are bacteria (e.g., E. coli).

Extraction of and Isolation of Cellular Components

Extraction of component molecules from cells typically begins by disruption of the cells outer membranes, cell walls, etc., thereby releasing the cell contents into the surrounding medium to form a “cell extract.” Detergents are widely used in the art to solubilize membranes, to enhance permeabilization effects of various chemical agents, and for disruption of the bacterial cell walls, facilitating the preparation of proteins, nucleic acids, etc., from cells. The term “detergent” as used herein refers to amphipathic surface-active agents (“surfactants”) that, when added to a liquid, reduce surface tension of the liquid in comparison to the same liquid in the absence of the detergent. See, e.g., Detergents: A guide to the properties and uses of detergents in biological systems, Calbiochem-Novabiochem Corporation, 2001, which is hereby incorporated by reference in its entirety.

The use of alkylphosphine oxide detergents, particularly in a near-physiological and non-alkaline environment, are provided herein. While the present invention relates to the use of APO detergents, the cellular extraction procedures described herein may also comprise the use of other, non-APO, detergents to assist in separation of the desired cellular components from their undesired counterparts. Cells to be extracted may be contacted with such other detergents before, during, and/or after the time when the cells to be extracted are contacted with the APO detergent. With regard to these other detergents, the following poly(ethylene oxide) detergents have the following formulas, and include, for example, NP-40, TRITON® X-100, TRITON® X114, C₁₂E₈, C₁₂E₉, GENAPOL® X-080, GENAPOL® X-100, LUBROL® PX, BRIJ® 35, TWEEN® 20, and TWEEN® 20:

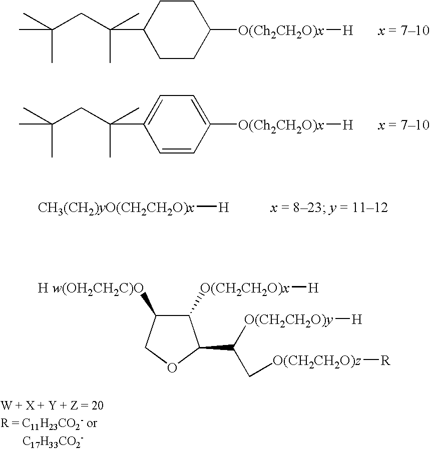

Alkyl glycosides often have the following formulas, and include dodecyl-β-D-maltoside (“dodecyl maltoside”), n-nonyl-β-D-glucopyranoside, n-octyl-β-D-glucopyranoside (“octyl glucoside”), n-heptyl-β-D-glucopyranoside, n-hexyl-β-D-glucopyranoside, and octyl-β-D-thioglucopyranoside:
R—O—(CH₂)ₓ—CH₃ R=glucose, maltose, lactose, xylose, galactose, x=5-16;
R—S—(CH₂)ₓ—CH₃ R=glucose, maltose, lactose, xylose, galactose, x=5-16.

Alkyl amine N-oxides often have the following formula and include lauryl dimethylamine oxide:

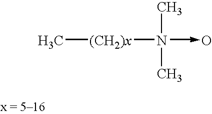

The use of physical methods of disruption, or a combination of chemical and physical approaches, may be combined with APO detergent treatment as required to provide a desired level of extraction. Chemical methods of disruption of the bacterial cell wall generally involve treatment of cells with organic solvents, kosmotropes, chaeotropes, antibiotics, detergents, and/or enzymes. When APO detergents are not employed during the initial steps of fractionation, they may be added subsequently in order to facilitate fractionation of the cell extract into various subportions. The addition of these various components to an extraction protocol are well known to those of skill in the art, and their inclusion is often made on an empirical basis in order to improve separation of desired molecules from undesired cellular material, and in order to retain the desired functional characteristics in the isolated molecule(s).

Detergents, including the APO detergents, may be used at concentrations ranging from about 0.02% w/v up to their solubility limit. Sometimes, the concentration of a particular detergent ranges from about 0.02% w/v to about 4% w/v, about 0.1% w/v to about 2% w/v, or is approximately about 0.5% to about 2% of the total solution.

The term “about” as used herein refers to +/−5% of a given value, often +/−2% of a given value, and sometimes the given value itself.

The APO detergent is sometimes used in a non-alkaline environment. As used herein, the term “non-alkaline” refers to a pH of less than 10. pH values sometimes lie between about 6 and about 9, and are sometimes near neutral (that is, between about pH 7 and about pH 7.5). If necessary, one or more buffering agents may be added to the APO detergent before, during, or after the detergent is placed in contact with cells to be extracted, or may be added to the cells before, during, or after the cells are contacted with the APO detergent. Numerous suitable buffering agents are known in the art to provide such non-alkaline environments. The following table provides an exemplary list of buffering agents and their associated pK values. Other suitable buffering agents are well known to those of skill in the art.

| | | Temperature | | | |
|---|---|---|---|---|---|
| Common | | Coefficient | | | |
| Name | Formula Weight | (↑pKa/° C.) | pKa(4° C.) | pKa(20° C.) | pKa(37° C.) |
| ACES | 182.19 | 0.020 | 7.20 | 6.90 | 6.54 |
| ADA | 190.16 | 0.011 | 6.80 | 6.60 | 6.43 |
| BES | 213.25 | 0.016 | 7.41 | 7.5 | 6.88 |
| Bicine | 163.17 | 0.018 | 8.64 | 8.35 | 8.04 |
| CHES | 207.29 | 0.011 | 9.73 | 9.55 | 9.36 |
| Glycylglycine | 132.12 | 0.028 | 8.85 | 8.40 | 7.92 |
| Glycinamide | 110.54 | 0.029 | 8.66 | 8.20 | 7.71 |
| HCL | | | | | |
| HEPES | 238.31 | 0.014 | 7.71 | 7.55 | 7.31 |
| HEPPS | 252.33 | 0.011 | 8.18 | 8.00 | 7.81 |
| MES | 213.26 | 0.011 | 6.33 | 6.15 | 5.96 |
| MOPS | 209.27 | 0.011 | 7.38 | 7.20 | 7.01 |
| PIPES | 302.37 | 0.0085 | 6.94 | 6.80 | 6.66 |
| TES | 229.25 | 0.020 | 7.82 | 7.50 | 7.16 |
| TRICINE | 179.17 | 0.021 | 8.49 | 8.15 | 7.79 |
| TRIS | 121.14 | 0.031 | 8.80 | 8.30 | 7.77 |

Because certain types of cells (e.g., mammalian cells) do not comprise a cell wall, they are particularly susceptible to osmotic lysis. Particularly gentle lysis and extraction methods may comprise the inclusion of one or more solutes to maintain an isoosmotic environment during detergent treatment. For these purposes, poly(ethylene glycol), glycerol, sorbitol, and dextrans are often used. The amount necessary to provide such an osmotically balanced solution will depend upon the cell type, and may be determined empirically by the artisan.

Certain reagents finding use in preparing cell extracts can depend upon the type of molecule to be isolated. For example, in some situations the use of an enzyme to hydrolyze the cell wall in order to release the proteins from the cell is particularly advantageous. One such enzyme that has proven to be very useful for bacterial cell wall lysis and protein extraction is lysozyme. Lysozyme (N-Acetylmuramyl Hydrolase Muramidase) is frequently used for bacterial lysis because of its ability to hydrolyze the b1,4 linkages between N-acetylmuramic acid (2-acetamido-2-deoxy-3-O-[lcarboxymethyl]-glucopyranose) residues in the bacterial cell wall. Another example is proteinase K, a nonspecific protease used during nucleic acid isolation to digest contaminating proteins present in the extract. A contraexample to proteinase K is the protease inhibitors often used in extracts to prevent digestion of proteins during their isolation.

Once a cell extract is obtained, further purification procedures are well known to those of skill in the art. See, e.g., Deutscher, Methods in Enzymology, Vol. 182, “Guide to Protein Purification,” 1990. Various precipitation, chromatographic, and/or electrophoretic methods may be employed to purify the polypeptide(s) and/or nucleic acid(s) of interest from the extract. These include differential centrifugation, precipitation by various means (e.g., using ammonium sulfate or polycations such as polyethylenimine), ion exchange chromatography (e.g., using DEAE, quarternary amine, phosphorl and/or carboxyl functionalities on cellulose, agarose or polymeric beads), affinity chromatography (e.g., heparin on agarose or polymeric beads), hydrophobic interaction chromatography (e.g., butyl, octyl or hexyl functionalities on agarose or polymeric beads), hydoxylapatite chromatography, size exclusion chromatography, etc. Chromatography may be performed using low pressures (e.g., gravity-driven flow), or at higher pressures (e.g., using instruments such as FPLC or HPLC).

In the present invention, “isolated” refers to material removed from its original environment (e.g., the natural environment if it is naturally occurring, or a host cell if expressed exogenously), and thus is altered “by the hand of man” from its original environment. The term “purified” as used herein with reference to molecules does not refer to absolute purity. Rather, “purified” is intended to refer to a substance in a composition that, if a polypeptide, contains fewer polypeptide species other than the polypeptide of interest in comparison to the organism from which it originated. “Purified,” if a nucleic acid, refers to a substance in a composition that contains fewer nucleic acid species other than the nucleic acid of interest in comparison to the organism from which it originated. Sometimes, a polypeptide or nucleic acid is “substantially pure,” indicating that the polypeptide or nucleic acid represents at least 50% of polypeptide or nucleic acid on a mass basis of the composition. Often, a substantially pure polypeptide or nucleic acid is at least 75% on a mass basis of the composition, and sometimes at least 95% on a mass basis of the composition.

In the case of polypeptides expressed recombinantly, a particularly advantageous method for isolation of a polypeptide of interest is to provide an affinity tag engineered to be expressed as a fusion with the polypeptide of interest. The following table shows the sequences of several common affinity tags:

| | |
|---|---|
| Tag | Amino Acid Sequence |

| | |
|---|---|
| His | HHHHHH |
| c-MYC | EQKLISEEDL |
| HA | YPYDVPDYA |
| VSV-G | YTDIEMNRLGK |
| HSV | QPELAPEDPED |
| V5 | GKPIPNPLLGLDST |
| FLAG | DYKDDDDKG |
| Lumio | CCPGCC |

Some of these tags are conveniently bound by “chelate” columns (Ni or arsenic chelate columns are commonly used) or by antibodies directed to the sequence. The cognate affinity partner for the tags often is conveniently provided on a solid support. The term “solid phase” as used herein refers to a wide variety of materials including solids, semi-solids, gels, films, membranes, meshes, felts, composites, particles, and the like typically used by those of skill in the art to sequester molecules. The solid phase can be non-porous or porous. Suitable solid phases include those developed and/or used as solid phases in solid phase binding assays. See, e.g., chapter 9 of Immunoassay, E. P. Diamandis and T. K. Christopoulos eds., Academic Press: New York, 1996, hereby incorporated by reference. Examples of suitable solid phases include membrane filters, cellulose-based papers, beads (including polymeric, latex and paramagnetic particles), glass, silicon wafers, microparticles, nanoparticles, TentaGels, AgroGels, PEGA gels, SPOCC gels, and multiple-well plates. See, e.g., Leon et al., Bioorg. Med. Chem. Lett. 8: 2997 (1998); Kessler et al., Agnew. Chem. Int. Ed. 40: 165 (2001); Smith et al., J. Comb. Med. 1: 326 (1999); Orain etal., Tetrahedron Lett. 42: 515 (2001); Papanikos et al., J. Am. Chem. Soc. 123: 2176 (2001); Gottschling et al., Bioorg. And Medicinal Chem. Lett. 11: 2997 (2001).

Overexpression of proteins in prokaryotes often leads to the production of insoluble aggregates of misfolded proteins in inclusion bodies. Often considered a nuisance, the formation of inclusion bodies has the advantage of a high enrichment of the desired protein at an early stage of purification. Furthermore, the recombinant protein is protected in inclusion bodies against proteolysis by intracellular proteases. These inclusion bodies can easily be purified and may be the best method for the production of proteins that are lethal to the host cells. However, the solubilization of the expressed protein often is obtained using strongly denaturing conditions. Since inclusion body proteins do not readily disintegrate under physiological conditions, the solubilization requires rather strong chaotropic agents such as 6 M guanidine hydrochloride or 6M-8 M urea. Guanidine hydrochloride is often preferred over urea because it may solubilize extremely sturdy inclusion bodies, and because urea solutions may contain isocyanate leading to carbamylation of the free amino groups of the polypeptide. In the case of proteins containing cysteine, the isolated inclusion bodies usually contain some interchain disulfide bonds which reduce the solubility. Addition of reducing agents, like DTT and/or 2-mercaptoethanol, in combination with chaotropic agents allows reduction of the interchain disulfide bonds.

Expression of Recombinant Polypeptides

While the compositions and methods of the present invention may be used to facilitate isolation of polypeptides endogenously produced by cells, they are particularly well suited to facilitate isolation and/or purification of recombinantly produced polypeptides. Recombinant expression of a polypeptide requires construction of an expression vector containing a polynucleotide that encodes the polypeptide. Methods which are well known to those skilled in the art can be used to construct expression vectors containing the necessary coding sequences and appropriate transcriptional and translational control signals. See, for example, the techniques described in Sambrook et al., Molecular Cloning, A Laboratory Manual, 2d Ed., Cold Spring Harbor Laboratory, Cold Spring Harbor, N.Y., 1990; and Ausubel et al., eds., Current Protocols in Molecular Biology, John Wiley & Sons, NY, 1998, which are both incorporated by reference herein in their entireties. These methods include, for example, in vitro recombinant DNA techniques, synthetic techniques, and in vivo genetic recombination.

The choice of expression vector depends on the intended host cells in which the vector is to be expressed. Typically, the vector includes a promoter and other regulatory sequences in operable linkage to the inserted coding sequences that ensure the expression of the latter. Use of an inducible promoter is advantageous to prevent expression of inserted sequences except under inducing conditions. Inducible promoters include arabinose, lacZ, metallothionein promoter or a heat shock promoter. Cultures of transformed organisms can be expanded under noninducing conditions without biasing the population for coding sequences whose expression products are better tolerated by the host cells. The vector may also provide a secretion signal sequence position to form a fusion protein with polypeptides encoded by inserted sequences, although often inserted polypeptides are linked to a signal sequences before inclusion in the vector. The expression vector is transferred to a host cell by conventional techniques and the transfected cells are then cultured by conventional techniques.

E. coli is one prokaryotic host useful particularly for cloning the polynucleotides of the present invention. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation.

Any convenient cloning strategy may be utilized to incorporate an element, such as a nucleotide sequence that encodes a protein or polypeptide, into a cloning or expression vector. Known methods can be utilized to insert an element into the vector, such as (1) cleaving the vector at one or more existing restriction enzyme sites and ligating an element of interest and (2) adding restriction enzyme sites to the template by hybridizing oligonucleotide primers that include one or more suitable restriction enzyme sites and amplifying by polymerase chain reaction (PCR). Other cloning strategies take advantage of one or more insertion sites present or inserted into the template nucleic acid.

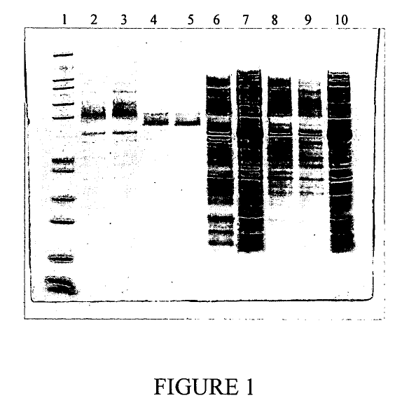

In some embodiments, a nucleic acid utilized for cloning and/or expression includes one or more recombinase insertion sites. A recombinase insertion site is a recognition sequence on a nucleic acid molecule that participates in an integration/recombination reaction by recombination proteins. For example, the recombination site for Cre recombinase is loxP, which is a 34 base pair sequence comprised of two 13 base pair inverted repeats (serving as the recombinase binding sites) flanking an 8 base pair core sequence (e.g., FIG. 1 of Sauer, B., Curr. Opin. Biotech. 5:521-527 (1994)). Other examples of recombination sites include attB, attP, attL, and attR sequences, and mutants, fragments, variants and derivatives thereof, which are recognized by the recombination protein λ Int and by the auxiliary proteins integration host factor (IHF), FIS and excisionase (Xis) (e.g., U.S. Pat. Nos. 5,888,732; 6,143,557; 6,171,861; 6,270,969; 6,277,608; and 6,720,140; U.S. patent aplication Ser. Nos. 09/517,466, filed Mar. 2, 2000, and 09/732,914, filed Aug. 14, 2003, and in U.S. patent publication no. 2002-0007051-A1; Landy, Curr. Opin. Biotech. 3:699-707 (1993)). Examples of recombinase cloning nucleic acids are in Gateway® systems (Invitrogen, Calif.), which include at least one recombination site to clone desired nucleic acid molecules in vivo or in vitro. In some embodiments, the system utilizes vectors that contain at least two different site-specific recombination sites, often based on the bacteriophage lambda system (e.g., att1 and att2), and are mutated from the wild-type (att0) sites. Each mutated site has a unique specificity for its cognate partner att site (i.e., its binding partner recombination site) of the same type (for example attB1 with attP1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are cloned and subcloned using the Gateway® system by replacing a selectable marker (for example, ccdB) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects.

In certain embodiments, a nucleic acid for cloning and/or expression includes one or more topoisomerase insertion sites. A topoisomerase insertion site is a defined nucleotide sequence recognized and bound by a site-specific topoisomerase. For example, the nucleotide sequence 5′-(C/T)CCTT-3′ is a topoisomerase recognition site bound specifically by most poxvirus topoisomerases, including vaccinia virus DNA topoisomerase I. After binding to the recognition sequence, the topoisomerase cleaves the strand at the 3′-most thymidine of the recognition site to produce a nucleotide sequence comprising 5′-(C/T)CCTT-PO₄-TOPO, a complex of the topoisomerase covalently bound to the 3′ phosphate via a tyrosine in the topoisomerase (e.g., Shuman, J. Biol. Chem. 266:11372-11379, 1991; Sekiguchi and Shuman, Nucl. Acids Res. 22:5360-5365, 1994; U.S. Pat. No. 5,766,891; PCT/US95/16099; and PCT/US98/12372). In comparison, the nucleotide sequence 5′-GCAACTT-3′ is a topoisomerase recognition site for type IA E. coli topoisomerase III. An element to be inserted often is combined with topoisomerase-reacted template and thereby incorporated into the template nucleic acid (e.g., http address www.invitrogen.com/downloads/F-13512_Topo_Flyer.pdf; http address at www.invitrogen.com/content/sfs/brochures/710_{—}021849%20_B_TOPOCloning_bro.pdf; TOPO TA Cloning® Kit and Zero Blunt® TOPO® Cloning Kit product information).

When a large quantity of a protein is to be produced, vectors which direct the expression of high levels of fusion protein products that are readily purified may be desirable. Such vectors include, but are not limited, to the E. coli expression vector pUR278 (Ruther et al., EMBO J. 2:1791 (1983)), in which the polypeptide coding sequence may be ligated individually into the vector in frame with the lac Z coding region so that a fusion protein is produced; pIN vectors (Inouye & Inouye, Nucleic Acids Res. 13:3101-3109 (1985); Van Heeke and Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety. Suitable cleavage sites for inclusion in fusion constructs include those in the following table.

| | Cleavage Enzyme/ | |
|---|---|---|
| Excision site ↓ | Self-Cleavage | Comments |
| Asp-Asp-Asp-Asp-Lys↓ | Enterokinase | The site will not cleave if followed by a proline |
| | | residue. |
| | | Secondary cleavage sites at other basic |
| | | residues, depending on conformation of protein |
| | | substrate. Active from pH 4.5 to 9.5 and |
| | | between 4° C. and 45° C. |
| Ile-Glu/Asp-Gly-Arg↓ | Factor Xa | Will not cleave if followed by proline and |
| | protease | arginine. Secondary cleavage sites following |
| | | Gly-Arg sequences. |
| Leu-Val-Pro-Arg↓Gly-Ser | Thrombin | Secondary cleavage sites. |
| Glu-Asn-Leu-Tyr-Phe- | TEV protease | Seven-residue recognition site, making it a |
| Gln↓Gly | | highly site-specific protease. Active over a |
| | | wide range of temperatures. Protease available |
| | | as a His-tag fusion, allowing for protease |
| | | removal after recombinant protein cleavage. |
| Leu-Glu-Val-Leu-Phe- | PreScission ™ | Genetically engineered form of human |
| Gln↓Gly-Pro | protease | rhinovirus 3C protease with a GST fusion, |
| | | allowing for facile cleavage and purification of |
| | | GST-tagged proteins along with protease |
| | | removal after recombinant protein cleavage. |
| | | Enables low-temperature cleavage of fusion |
| | | proteins containing the eight-residue |
| | | recognition sequence. |

Other microbes, such as yeast, are also be used for expression. Saccharomyces is a preferred host, with suitable vectors having expression control sequences, such as promoters, including 3-phosphoglycerate kinase or other glycolytic enzymes, and an origin of replication, termination sequences and the like as desired.

Mammalian tissue cell culture can also be used to express and produce the polypeptides of the present invention (see Winnacker, From Genes to Clones (VCH Publishers, N.Y., N.Y., 1987). A number of suitable host cell lines capable of secreting polypeptides have been developed including insect cells for baculovirus expression, CHO cell lines, various Cos cell lines, HeLa cells, myeloma cell lines, transformed B-cells and hybridomas. Expression vectors for these cells can include expression control sequences, such as an origin of replication, a promoter, and an enhancer (Queen et al., Immunol. Rev. 89: 49-68 (1986)), and necessary processing information sites, such as ribosome binding sites, RNA splice sites, polyadenylation sites, and transcriptional terminator sequences. Expression control sequences sometimes are promoters derived from immunoglobulin genes, SV40, adenovirus, bovine papilloma virus, or cytomegalovirus.

In addition, a host cell strain may be chosen, which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells, which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, and in particular, breast cancer cell lines such as, for example, BT483, Hs578T, HTB2, BT20 and T47D, and normal mammary gland cell line such as, for example, CRL7030 and Hs578Bst.

Methods for introducing vectors containing the polynucleotide sequences of interest vary depending on the type of cellular host. For example, calcium chloride transfection is commonly utilized for prokaryotic cells, whereas calcium phosphate treatment or electroporation may be used for other cellular hosts. (See generally Sambrook et al., supra).

### EXAMPLES

The following examples serve to illustrate the present invention. These examples do not limit the scope of the invention.

### Example 1

### Reagents

The following reagents were used in the following Examples:

| Material | Cat# | Vendor |
|---|---|---|
| n-Octyl-b-D-Glucopyranoside (OGP) | 494459 | Calbiochem |
| n-Nonyl-b-D-Glucopyranoside | 488285 | Calbiochem |
| n-Octyl-b-D-Thioglucopyranoside | 494461 | Calbiochem |
| Dimethyloctylphosphine oxide (APO-8) | KP3001 | Calbiochem |
| Dimethylnonylphosphine oxide (APO-9) | KP3002 | Calbiochem |
| Dimethyldecylphosphine oxide (APO-10) | KP3003 | Calbiochem |
| Dimethylmonodecylphosphine oxide (APO-11) | KR3004 | Calbiochem |
| Dimethyldodecylphosphine oxide (APO-12) | KR3005 | Calbiochem |
| Lumio ™ Green Detection Kit | LC6090 | Invitrogen |
| InVision ™ His-tag In-gel stain | LC6030 | Invitrogen |
| Triton X-100 | X100 | Sigma |
| IGEPAL-630 | 1-3021 | Sigma |
| Elugent | 324707 | Calbiochem |
| Tris | T87602 | Sigma |
| NaCl | 433209 | Aldrich |
| Na phosphate, monobasic | 331988 | Aldrich |
| BugBuster ® HT | 70922 | Novagen |
| BugBuster ® | 70584 | Novagen |
| B-PER | 78248 | PIERCE |
| B-PER II | 78260 | PIERCE |
| 4-12% NuPAGE ® Bis-Tris gels, 1 mm, | NP0321 | Invitrogen |
| 10 wells | | |
| 20x MES SDS Running Buffer | NP0002 | Invitrogen |
| BL21-AI ™ competent cells | C6070-03 | Invitrogen |
| BL21 Star ™ competent cells | C6010-03 | Invitrogen |
| BL21(DE3) competent cells | C6000-03 | Invitrogen |
| BL21(DE3)pLysS competent cells | C6020-03 | Invitrogen |

### Example 2

### Expression of Polypeptides

E. coli strain BL21 cells were transformed with plasmids expressing β-galactosidase, green fluorescent protein (GFP) or maltose binding protein (MBP) as follows. BL21-AI were transformed with pET 100 expressing n-terminal 6×His tagged GFP or n-terminally 6×His tagged LacZ (β-galactosidase). The cells were grown at 37° C. to OD₆₀₀=0.4-0.5 and then induced with 1 mM IPTG and 0.1% arabinose. The expression was performed for 2-4 hours at 37° C. BL21 Star cells were transformed with plasmid expressing MBP. The cells were grown at 37° C. to OD₆₀₀=0.4-0.5 and then induced with 1 mM IPTG. Protein expression was performed at 30° C. for 2-4 hours. BL-21(DE3) and BL-21(DE3)pLysS cells were transformed with vector expressing N-terminally 6×His tagged LacZ (β-galactosidase) (pET 100 lacZ-IVGN cat# K100-01), N-terminal Lumio tag (Cys-Cys-Pro-Gly-Cys-Cys) and 6×His tagged Lac-Z (pET 160 LacZ), and N-terminal Lumio tag (Cys-Cys-Pro-Gly-Cys-Cys) and 6×His tagged GFP (pET 160 GFP). Cultures were grown at 37° C. to OD₆₀₀ of 0.4-0.5, then induced by adding IPTG to a final concentration of 1 mM. Expression was performed at 37° C. for 3 hours.

Cultures were grown to OD₆₀₀=1.2-1.5 and then collected for lysis. One ml of culture was subjected to centrifugation (10,000×g) and lysed in 75 to 100 μl of each lysis buffer depending on optical density (OD). In some samples 10 μl of 1:100 dilution of BENZONASE® nuclease (Benzon Pharma A/S) and 10 μl of 0.5 mg/ml Lysozyme were added. Samples were allowed to incubate at room temperature for approximately 10 minutes, then spun down at 10,000×g. Supernatant was pipetted and used for subsequent activity assays and SDS-PAGE analysis. For BL21 (DE3) pLysS cultures, 2 independent lysates of each culture with and without lysozyme were prepared and tested.

### Example 3

### Data Collection and Reduction

GFP activity was determined by reading fluorescence (Excitation at 395/Emission 405) of lysates (diluted 1:20 or 1:40 in 50 mM Phosphate pH 7). The raw data was recorded. LacZ activity was determined by using the β-galactosidase assay kit (IVGN cat # K1455-01). The lysate samples were diluted 1:100 and then analyzed as described in the kit manual. Results were obtained using OD₄₂₀ readings in the following equation:
nmoles of ONPG hydrolyzed/μL lysate=(OD₄₂₀background) (1.92×10⁵ nl)(dilution factor=100) (4500 nl/nmoles-cm)×(1 cm)
Data was normalized to the reading received from a control sample prepared with BugBuster® or BugBuster® HT (Novagen) lysis reagents.

For gel electrophoresis, 15 μL of each lysate was incubated with 5 μl of 4× LDS Sample buffer and 2 μl of NuPage Reducing reagent in a 70° C. heat block for 10 minutes. 5 μl of the samples were run on 4-12% NuPAGE gels (Invitrogen cat # NP0321 or NP0323) with MES SDS running buffer. SDS-PAGE gels were stained with SimplyBlue Safe Stain (Invitrogen cat# LC6065).

For His-tagged β-galactosidase collection, 50 ml cell culture was dispensed in a 50 ml centrifuge tube, and centrifuged at 10,000×g for 5 minutes. The collected medium was discarded and 8 ml of 0.5% APO-10 lysis buffer were added. Then 80 μL of 5 mg/mL lysozyme was added to the sample, and 8 μL of BENZONASE® nuclease (Benzon Pharma A/S) was added to decrease the viscosity of the sample. The sample was vortexed until the pellet was resuspended, and then incubated at room temperature for 10 minutes. The tube was centrifuged at 10,000×g for 5 minutes. The soluble lysate was added to 2 ml resin in a nickel chelate resin column, and agitated for I hour. The resin was washed 4 times with Native Wash Buffer. The μ-galactosidase was eluted with 8 ml of Native Elution Buffer containing 500 mM imidazole. Eight 1 mL fractions were collected from the sample.

Lumio® Green In-Gel detection kit LC6090 was used for the detection of the Lumio tag. 15 μL of each sample was mixed with 5 μL of Lumio Sample Buffer. 0.2 μL of Lumio Green detection reagent was added to each sample and the samples were incubated for 10 minutes at 70° C. Then 2 μL of the Lumio Enhancer was added to each sample and incubated for 5 minutes at room temperature. 5 μL of each sample was loaded on 4-12% NuPage BisTris gel. Detection was performed on an Alpha Imager 3000 with excitation at 302 nm and using a fluorescein filter.

For InVision® His-tag detection, 15 μL of each sample was mixed with 5 μL of 4×LDS Sample Buffer and 2 μL NuPAGE reducing reagent, and heated for 10 minutes at 70° C. Then 5 μL was loaded on 4-12% NuPAGE Bis-Tris gel. After the run the gel was detected as described in the InVision® His-tag stain product insert.

### Example 4

### Initial Screening of Detergents for Lysis and Protein Recovery

Screening of a wide range of detergents was performed by evaluating the lysis of uninduced cell cultures. For the first round of screening, detergents from different chemical groups were evaluated starting with some well-known detergents such as Triton X-100, Elugent, NP-7, NP-40. The lysis buffers were prepared at 1% detergent concentration in 50 mM phosphate pH 7. The detergents were compared with known lysis procedures using BugBuster® lysis reagent (Novagen), B-Per™ protein extraction reagent (Pierce), Relay 96 Protein Screen (Invitrogen), and sonication. Triton X-100, NP-7, NP-40 and Elugent were determined to not be very efficient for lysing bacterial cells. Significantly less protein was seen in these samples as compared to BugBuster® lysis reagent (Novagen) and B-Per™ protein extraction reagent (Pierce).

Next, a number of different alkyl glycoside detergents (1% of n-octyl-β-D-glucopyranoside, n-octyl-β-D-thioglucopyranoside, n-Nonyl-β-D-glucopyranoside, and n-dodecyl-β-D-maldoside in 50 mM phosphate, 300 mM NaCl pH 7) were tested and showed good lysis performance, which was comparable to what was observed with BugBuster® lysis reagent (Novagen).

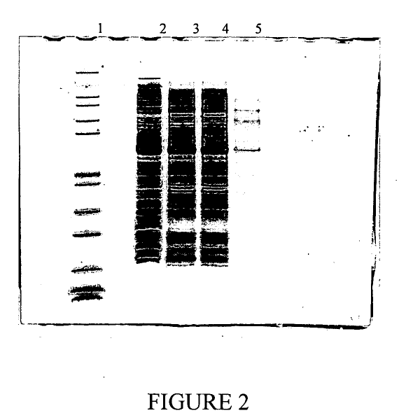

Other types of detergents also screened were ASB-C8 and three other synthesized sulfobetaine detergents: SB-A, SB-B, and SB-C, as well as APO-8, APO-10, APO-12 and N-dodecanoyl sucrose as 1% detergent solutions in 50 mM phosphate, 300 mM NaCl pH 7) using BL21 Star cells. FIG. 1 shows lysis of uninduced BL21 Star cells with detergents by PAGE. Lane 1 is a Mark 12 Molecular Weight Standard; lane 2 is a 1% SB-A lysis solution; lane 3 is a 1% ASB-C8 lysis solution; lane 4 is a 1% SB-B lysis solution; lane 5 is a 1% SB-C lysis solution; lane 6 is a 1% APO-8 lysis solution; lane 7 is a 1% APO-10 lysis solution; lane 8 is a 1% APO-12 lysis solution; lane 9 is a 1% N-Dodecanoyl Sucrose lysis solution and lane 10 is BugBuster® HT lysis reagent. As shown in FIG. 1 only the APO-10 detergent performed very well. The other detergents including APO-8 and APO-12 did not lyse the cells efficiently. It is likely that the length of the alkyl chain is critical for the lysis ability of the detergent. Therefore, another round of testing was performed with detergents of the same type with alkyl chains one carbonyl smaller and bigger than APO-10. FIG. 2 shows the results of the testing with APO-9, APO-10 and APO-11 detergents. One percent detergent formulations were prepared in 50 mM phosphate, 300 mM NaCl pH=7. In FIG. 2, Lane 1 is a Mark 12 Molecular Weight Standard; lane 2 is BugBuster® HT; lane 3 is a 1% APO-9 lysis solution; lane 4 is a 1% APO-10 lysis solution and lane 5 is a 1% APO-11 lysis solution. As shown in FIG. 2, the optimal lysis capabilities for the phosphine based detergents lay in the 9-10 carbon atoms of the alkyl chain.

Side by side comparison testing was performed of the alkylglucoside and alkylphosphine detergents with several different cell types: BL-21-AI, BL-21-Star, BL-21 DE3. In this comparison testing BugBuster® HT lysis reagent (Novagen), B-Per™ protein extraction reagent (Pierce), n-octyl-β-D-glucopyranoside, n-nonyl-β-D-glucopyranoside, and n-octyl-β-D-thioglucopyranoside, APO-9 and APO-10 were utilized. All of these detergents performed well as lysis reagents with the best performing detergents being the n-nonyl-β-D-glucopyranoside and n-octyl-β-D-thioglucopyranoside. Further optimization and testing was concentrated on the use of APO detergents by themselves and in combination with other detergents.

### Example 5

### Screening of APO Detergents

Following preliminary screening, more detailed testing was performed using GFP, β-galactosidase and MBP expressed in E. coli. The GFP and β-galactosidase proteins were selected because they provided a way to assess the activity (native state) of the proteins. The activity of the β-galactosidase was measured using the β-galactosidase activity kit (Invitrogen) as described above. The GFP activity (native state) was tested by measuring the fluorescence of the protein as described above. MBP extraction was evaluated qualitatively by SDS PAGE gel electrophoresis.

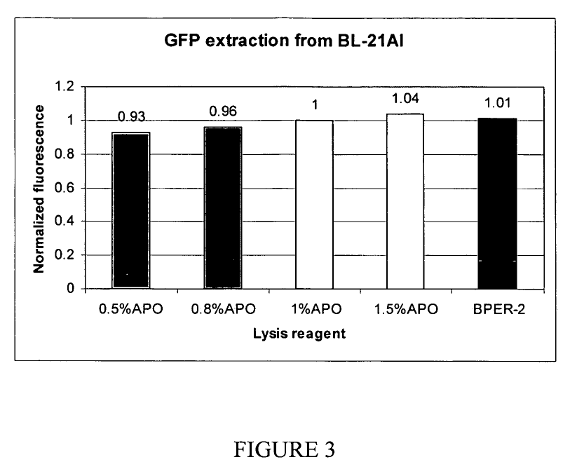

As GFP is fluorescent in its native state and loses its fluorescence when denatured, it was utilized to conveniently determine which lysis reagents preserved the native state of proteins. The following lysis buffer formulations were tested side by side with B-PER II and BugBuster®: 0.5% APO-10 in 50 mM Tris pH=7; 0.8% APO-10 in 50 mM Tris pH=7; 1% APO-10 in 50 mM Tris pH=7 and 1.5% APO-10 in 50 mM Tris pH=7. FIG. 3 shows results of lysing BL21-AI cells expressing GFP with these detergents in multiple treatments. The results were normalized to the reading for the BugBuster® reagent as described above and the chart shows the average of results obtained for each lysis reagent. The results achieved with the lowest concentration (0.5%) of APO detergent lysis reagent were within 7% from the results achieved with the BugBuster® reagent. There was some variation in the results from experiment to experiment. While the 0.5% APO reagent sometimes was more effective than the BugBuster® reagent and sometimes the BugBuster® reagent was more effective, differences in the effectiveness between the two reagents were within the margin of error.

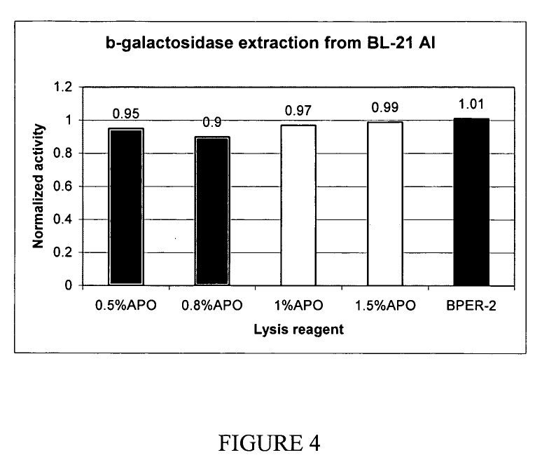

Beta-galactosidase was selected for testing as it also allowed for a convenient assessment of effects of lysis conditions on protein structure. FIG. 4 shows the performance of various concentrations of APO-10 detergent used for lysis of BL21- AI cells expressing β-galactosidase. FIG. 4 shows the performance of the 0.5% APO detergent formulation was within 5% of the performance of the BPER-2 reagent. The extraction of the P-galactosidase was performed using lysozyme in the lysis buffer. None of the lysis reagents tested extracted significant amounts of β-galactosidase without also lysing the cell wall with lysozyme. Stated another way, there was almost no β-galactosidase present in the lysate when no lysozyme was present in the lysis buffer. Without being limited by theory, this effect may be due to non-ionic detergent lysis reagents introducing holes in the cell wall without completely disintegrating the walls. The holes are likely wide enough for smaller proteins to leak out of the cells but not the bigger proteins. Confirmation of this assessment is there were no proteins larger than about 110 kDa present in the lysates without lysozyme. The extraction efficiency for proteins larger than about 80 kDa decreased when no lysozyme was included in the lysis buffer. Thus, lysozyme often is included in a lysis when proteins larger than 70-80 kDa are expressed. For smaller proteins the inclusion of lysozyme is optional as it does not affect the amount of protein extracted. No lysozyme is required when lysozyme-expressing strains of E. coli, such as pLysS, are utilized.

MBP extraction from BL21 Star cultures was comparable for lysis detergents tested (i.e., APO-10, BugBuster® HT, BPER-2 and OGP). APO-10 performed similarly or slightly better than the other detergents tested.

Compatibility of alkyl phosphine detergents with affinity purification resins and protein detection reagents also was assessed. Beta-galactosidase was purified using ProBond™ and NiNTA resins in the presence of APO-10 detergent without interference by the detergent. Beta-galactosidase was detected by an InVision™ protein detection agent and Chloramphenicol Acetyl Transferase (CAT) was detected by a Lumio™ protein detection agent in the presence of APO-10 detergent without interference from the detergent. Thus, an alkyl phosphine detergent was compatible with protein purification solid phases and protein detection agents.

The entirety of each patent, patent application, publication and document referenced herein hereby is incorporated by reference, including all tables, drawings, and figures. All patents and publications are herein incorporated by reference to the same extent as if each was specifically and individually indicated to be incorporated by reference. Citation of the above patents, patent applications, publications and documents is not an admission that any of the foregoing is pertinent prior art, nor does it constitute any admission as to the contents or date of these publications or documents. All patents and publications mentioned herein are indicative of the skill levels of those of ordinary skill in the art to which the invention pertains.

Modifications may be made to the foregoing without departing from the scope, spirit and basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. One skilled in the art readily appreciates that the present invention is well adapted to carry out the objects and obtain the ends and advantages mentioned, as well as those inherent therein. The examples provided herein are representative of specific embodiments, are exemplary, and are not intended as limitations on the scope of the invention.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

What is claimed is:

1. An aqueous solution which comprises a detergent and a buffer component at about pH 3 to about pH 10, wherein the detergent is of the following structure: 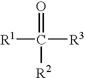
wherein R¹ and R² are independently selected from the group consisting of methyl, ethyl, 1-propyl, 2-propyl, 1-butyl, 2-butyl, 2-methyl-1-propyl, tert-butyl, 1 -pentyl, 2-pentyl, 3-pentyl, hydroxymethyl, 2-hydroxyethyl, 2-hydroxypropyl, 3-hydroxypropyl and 2,3-dihydroxypropyl; and R³ is a C8 to C16 linear or branched alkyl moiety.

2. The aqueous solution of claim 1, wherein R1 and R2 are methyl.

3. The aqueous solution of claim 1, wherein R3 is a C9 or C10 linear alkyl moiety.

4. The aqueous solution of claim 1, wherein R3 is a C10 linear alkyl moiety.

5. The aqueous solution of claim 1, wherein the pH is about 6 to about 9.

6. The aqueous solution of claim 5, wherein the pH is about 6.5 to about 7.5.

7. The aqueous solution of claim 1, wherein the detergent is about 0.2% to about 4% by weight in the solution.

8. The aqueous solution of claim 7, wherein the detergent is about 0.5% to about 2% by weight in the solution.

9. The aqueous solution of claim 1, wherein the buffer component is selected from the group consisting of a Tris buffer, a BisTris buffer, a phosphate buffer, a MOPS buffer, a MOPSO buffer, a PIPPS buffer and a HEPES buffer.

10. The aqueous solution of claim 1, which comprises a denaturant.

11. The aqueous solution of claim 10, wherein the denaturant is urea or sodium dodecyl sulfate.

12. The aqueous solution of claim 1, which comprises an agent that degrades a cell component.

13. The aqueous solution of claim 12, wherein the agent degrades cell walls.

14. The aqueous solution of claim 13, wherein the agent is lysozyme.

15. The aqueous solution of claim 12, wherein the agent degrades a nucleic acid.

16. The aqueous solution of claim 15, wherein the agent is a DNAse or RNAse.

17. The aqueous solution of claim 12, wherein the agent degrades an oligosaccharide.

18. The aqueous solution of claim 1, which comprises one or more salts.

19. The aqueous solution of claim 1, which comprises one or more other detergents.

20. The aqueous solution of claim 19, wherein one of the one or more other detergents is a Tween detergent or Triton X-100 detergent.

21. The aqueous solution of claim 1, which comprises a viscosogen or osmolyte agent.

22. The aqueous solution of claim 21, wherein the agent is glycerol or sucrose.

23. The aqueous solution of claim 1, which comprises a reducing agent.

24. The aqueous solution of claim 23, wherein the reducing agent is a thiol reducing agent.

25. The aqueous solution of claim 1, which comprises a protein detection agent or protein capture agent.

26. The aqueous solution of claim 25, wherein the detection agent or capture agent comprises an arsenic atom or metal atom.

27. The aqueous solution of claim 26, wherein the detection agent or capture agent comprises 4′,5′-bis(1,3,2-dithioarsolan-2-yl)fluorescein-(1,2-ethanedithiol)₂.

28. The aqueous solution of claim 26, wherein the detection agent or capture agent comprises copper, zinc, nickel, or cobalt.

29. The aqueous solution of claim 25, wherein the capture agent is in association with a solid phase.

30. A kit for extracting a peptide or protein from cells which comprises a container that includes a detergent of the following structure: 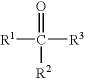
wherein R¹ and R² are independently selected from the group consisting of methyl, ethyl, 1-propyl, 2-propyl, 1-butyl, 2-butyl, 2-methyl-1-propyl, tert-butyl, 1-pentyl, 2-pentyl, 3-pentyl, hydroxymethyl, 2-hydroxyethyl, 2-hydroxypropyl, 3-hydroxypropyl and 2,3-dihydroxypropyl; and R³ is a C8 to C16 linear or branched alkyl moiety.

31. A method for extracting a peptide or protein from cells, which comprises: contacting the cells with a detergent in a solution at about pH 3 to about pH 10, wherein the detergent is of the following structure: 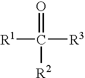
wherein R1 and R2 are independently selected from the group consisting of methyl, ethyl, 1-propyl, 2-propyl, 1-butyl, 2-butyl, 2-methyl-1-propyl, tert-butyl, 1-pentyl, 2-pentyl, 3-pentyl, hydroxymethyl, 2-hydroxyethyl, 2-hydroxypropyl, 3-hydroxypropyl and 2,3-dihydroxypropyl; and R3 is a C8 to C16 linear or branched alkyl moiety; whereby protein is extracted from the cells.

32. The method of claim 31, which further comprises isolating the peptide or protein.
